(19) United States
(12) Patent Application Publication
(10) Pub. No.: US 2009/0298147 A1
(43) Pub. Date: Dec. 3, 2009

(54) Method of Making (+)- Sitophilure

(76) Inventors: Dimitris Kalaitzakis, Crete (GR); J. David Rozzeli, Burbank, CA (US); Spiros Kambourakis, Pasadena, CA (US); Ioulia Smonou, Crete (GR)

Correspondence Address: CHRISTIE, PARKER & HALE, LLP, PO BOX 7068, PASADENA, CA, 91109-7068, US

(21) Appl. No.: 12/087,021

(22) PCT Filed: Dec. 22, 2006
(86) PCT No.: PCT/US06/49225

§ 371 Date: Mar. 3, 2009

Related U.S. Application Data
(60) Provisional application No. 60/753,877, filed on Dec. 23, 2005.

(51) Int. Cl. C12P 7/26

(52) U.S. Cl. 435/148

(57) ABSTRACT

(+)-Sitophilure, the aggregation pheromone of the pests rice weevil and maize weevil, is synthesized in high yield and diastereomeric excess by contacting 4-methyl-3,5heptadione with a reduced nicotinamide cofactor and a ketoreductase enzyme capable of catalyzing the reduction of 4-methyl-3,5-heptadione to produce (4R,5S)-5-hydroxy-4-methyl-3-heptanone to the substantial exclusion of other diastereomers.

### CROSS-REFERENCE TO RELATED APPLICATION

This application claims the priority benefit of U.S. Patent Application No. 60/753,877, filed Dec. 23, 2005, the contents of which are incorporated herein by this reference.

### FIELD OF TEE INVENTION

The invention relates to the synthesis of the pest pheromone, (+)-sitophilure, using enzymatic methods.

### BACKGROUND OF THE INVENTION

Optically active α-alkyl-β-hydroxy ketones are very important compounds in asymmetric organic synthesis because of their presence, as building blocks, in many natural products and pharmaceuticalse^{[i]}. Several methods have been developed for their synthesis^{[ii]}, including the stereoselective reduction of the corresponding α-alkyl-1,3-diketone using chiral chemical catalysts^{[iii]} or whole cell microbial catalysts^{[iv]}. Recently we published the stereoselective reduction of α-alkyl-1,3-diketones^{[iii]} utilizing twenty different isolated, NADPH-dependent ketoreductases^{[vi]}, and this method was proven to be very efficient for the synthesis of various optically and chemically pure α-alkyl-β-hydroxy ketones. Isolated ketoreductases offer many advantages compared to chemical or whole cell biocatalytic reductions and have been utilized for the stereoselective reduction of a variety of ketones^{[7]}. Scaling of successful enzymatic reductions to preparative scale (5-50 g) and high titers of ketone (0.7 M to 1.4 M) is usually straightforward and requires catalytic amounts of ketoreductase and cofactor^{[7a]}. [i] a) L. J. Vicario, D. Badia, E. Dominguez, M. Rodriguez, L. Carrillo, J. Org. Chem. 2000, 65, 3754; b) A. N. Van Draanen, S. Arseniyadis, T. M. Crimmins, H. C. Heathcock, J. Org. Chem. 1991, 56, 2499; c) H-X. Wei, R. L. Jasoni, H. Shao, J. Hu, P. W. Parè, Tetrahedron 2004, 60, 11829; d) A. D. Evans, J. V. Nelson, E. Vogel, T. R. Taber, J. Am. Chem. Soc. 1981, 103, 3099; e) Y. Yamamoto, K. Maruyama, Tetrahedron Leut. 1980, 21, 4607; f) C. H. Heathcock, C. T. Buse, W. A. Kleschick, M. C. Pirrung, J. E. Sohn, J. Lampe, J. Org. Chem. 1980, 45, 1066-1081; g) A. J. Marshall, M. M. Yanik, Org. Lett. 2000, 2, 2173; h) H-J. Li, H-Y. Tian, Y-C. Wu, Y-J. Chen, L. Liu, D. Wang, C-J. Li, Adv. Synth. Catal. 2005, 347, 1247; i) J. G. Solsona, J. Nebot, P. Romea, F. Urpi, J. Org. Chem. 2005, 70, 6533.[ii] a) F. Eustache, I. P. Dalko, J. Cossy, Org. Lett. 2002, 4, 1263; b) T. Yamada, T. Nagata, K, D. Sugi, K. Yorozu, T. Ikeno, Y. Ohtsuka, D. Miyazaki, T. Mukaiyama, Chem. Eur. J. 2003, 9, 4485.[iii] D. Kalaitzakis, J. D. Rozell, S. Kambourakis, I. Smonou, Org. Lett. 2005, 7, 4799.

In 1984, Burkholder and coworkers isolated the male-produced aggregation pheromone of the pests rice weevil (Sitophilus oryzae L.) and maize weevil (Sitophilus zeamais M.), which is named Sitophilure.^{[8]} This biologically-active compound was first identified as (4R,5S)-5-hydroxy-4-methyl-3-heptanone, from the extracts of thousands of insects. All four stereoisomers of this pheromone were synthesized and it was proved that the active form of this compound is the (4S,5R) enantiomer.^{[9]} Since then, several total syntheses of racemic,^{[10]} or other stereoisomers^{[11]} and the natural form^{[12]} of this pheromone have been published.

Serious economic losses of stored cereal grains (maize, rice and grain) are mainly caused by three weevils of the genus Sitophilus (Sitophilus zeamais, Sitophilus oryzae, Sitophilus granarius respectively) throughout the world. Early detection of infestations is critical in order to avoid further damage to the grains and the subsequent economic losses. Traps that contain very small amounts of synthetic (+)-sitophilure have been shown to be very effective in the early detection of all three species of weevils,^{[13]} however a simple scalable and economic method for the synthesis of this weevil attractant pheromone is still lacking.^{[9-12]} As a result, all of the commercially available traps for the early detection of these weevils are food-based.

### SUMMARY OF THE INVENTION

The present invention provides a simple, scalable, chemo-enzymatic synthesis of the natural pheromone (4R,5S)-5-hydroxy-4-methyl-3-heptanone, commonly known as (+)-Sitophilure, or “Sitophinone”; CAS No. 115014-45-4. The key step of this synthesis relies on the stereoselective reduction of a precursor of (+)-Sitophilure, 4-methyl-3,5-heptadione, by an isolated enzyme, e.g., NADPH-dependent ketoreductase, (KRED-A1C, sometimes referred to as “KRED-1-EXP-AIC”, Table 1) in the presence of a glucose/glucose dehydrogenase system for cofactor recycling (Scheme 1).

### DETAILED DESCRIPTION

In a first embodiment of the invention, (+) sitophilure is produced by contacting 4-methyl-3,5-heptadione with a reduced nicotinamide cofactor and a ketoreductase enzyme capable of catalyzing the reduction of 4-methyl-3,5-heptadione to produce (4R,5S)-5-hydroxy-4-methyl-3-heptanone to the substantial exclusion of other diastereomers. Preferably, the nicotinamide cofactor is NADPH, and preferably the cofactor is recycled during the synthesis. In a second embodiment, the method further comprises the step of producing 4-methyl-3,5-heptadione by methylating 3,5-heptadione.

Since Sitophilure is an optically active keto alcohol, it can be easily produced by the stereoselective reduction of the corresponding diketone 4-methyl-3,5-heptanedione, 1 (Scheme 1). Diketone 1 is also a naturally occurring aggregation pheromone of Sitona lineatous^{[14]}.

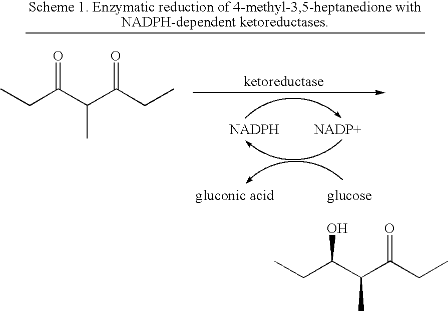

Per the invention, (+)-Sitophilure is produced to the substantial exclusion of other diastereomers. That is, the desired diastereomer is produced in greater than 80% diastereomeric excess (de), preferably, greater than 90% de, more preferably, greater than 98% de.

Positive enzymes for the reduction of 4-methyl-3,5-heptadione were identified after the screening of 64 isolated commercially available ketoreductases.^{[6]} Among them, three ketoreductases successfully produced (+)-sitophilure with high diastereo- and enantioselectivity. Many enzymes showed activity towards the reduction of 4-methyl-3,5-heptadione, and the best results of these enzymatic reductions are shown in Table 1. Note that all enzymes selectively produced the keto alcohol and not the diol even after longer incubation times.

TABLE 1
Enzyme-catalyzed stereoselective reduction of 4-methyl-3,5-heptanedione.

| | | Diastereomeric ratio | | | | | |
|---|---|---|---|---|---|---|---|
| | | % | | | | Conversion | |
| Substrate | KRED | A | B | C | D | (time) | Product |
| 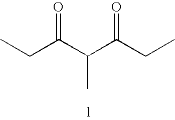 | 101 114 115 118 119 123 128 130 A1A A1B A1C A1D | 3 8 4 4 <1 20 3 6 <2 — — — | — 4 — — — — — — — 97 98 97 | 6 — 4 — — — 1 — — 3 2 3 | 91 88 92 96 >99 80 96 94 >98 — — — | 100% (6 h) 90% (24 h) >99% (6 h) 93% (24 h) 100% (12 h) 100% (6 h) 16% (24 h) 28% (24 h) 20% (24 h) 100% (40 min) 100% (1 h) 100% (1 h) | 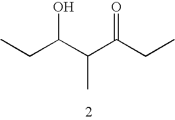 |

It is clearly demonstrated that two out the four stereoisomers, B and D, of the 5-hydroxy-4-methyl-3-heptanone are formed in optically pure form using five different enzymes, in very short reaction time, without the formation of the corresponding diol. In particular KRED-119 completed the reaction in 12 hours forming diastereomer D, whereas KRED-A1B, KRED-A1C and KRED-A1D completed the reaction in only 40 and 60 minutes respectively, forming the diastereomer B. To the best of our knowledge there is no other biocatalyst or chemical catalyst that can produce this keto alcohol in optically pure form, from the corresponding diketone, in such a short reaction time. The diastereomeric ratio and reaction time, presented in Table 1, were derived from chiral GC analysis.

In order to determine the absolute configuration of the two stereoisomers B and D we accomplished larger scale reductions and isolated keto alcohol-2, in high yield and optical purity (87% yield, 99% de, >99% ce with KRED-119 and 85% yield, 96% de, >99% ce with KRED-A1C). The ¹H-NMR^{[15]} and ¹³C-NMR^{[16]} of the isolated products indicate that the relative stereochemistry of the product D (Table 1) is anti and that of the product B is syn. The absolute stereochemistry of these hydroxy ketones was determined by assigning first the stereochemistry of the hydroxyl group by using chiral derivatizing agents.^{[17]} Therefore by using α-methoxyphenylacetic acid (MPA) the stereoisomers B and D were transformed into the corresponding MPA-esters (Scheme 2). The absolute stereochemistry of the enantiomers B and D (Scheme 3) was found to be (4S,5R) and (4S,5S) respectively, taking into account that the relative stereochemistry of the product D is anti and of the product B syn.

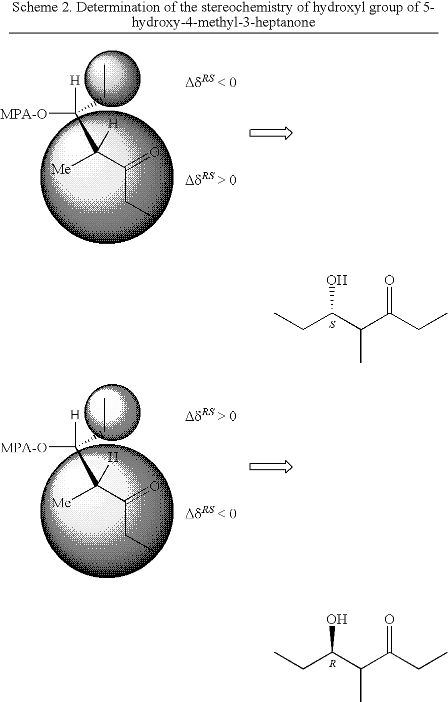

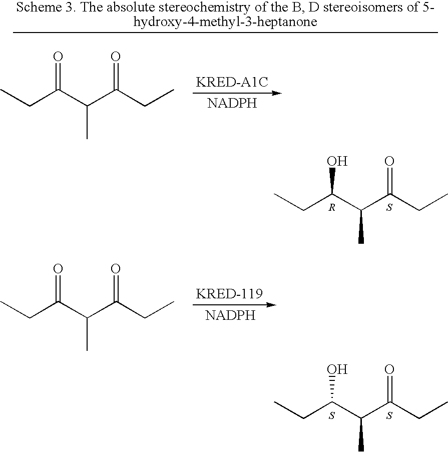

As we can see in Scheme 3, the product from the reduction of 4-methyl-3,5-heptanedione with KRED-A1C has the same stereochemistry with that of the natural pheromone (+)-Sitophilure. These results clearly indicate that ketoreductases KRED-A1B, KRED-A1C and KRED-A1D showed unusual anti-Prelog selectivity, concerning reduction of the 5-keto group and successfully produced the keto alcohol with the desired stereochemistry 4S,5R. So the natural product can be produced easily from the corresponding diketone.

In large scale, the reaction is completed in 24 hours, producing the pheromone with chemical yield 85%, de 96%, ee >99%, and chemical purity >99%, utilizing catalytic amounts of the NADPH cofactor (0.81% relative to the substrate), which was recycled in situ using Glucose Dehydrogenase (GDH). The corresponding 4-methyl-3,5-heptanedione can be readily produced from the commercially available 3,5-heptanedione (Scheme 4).

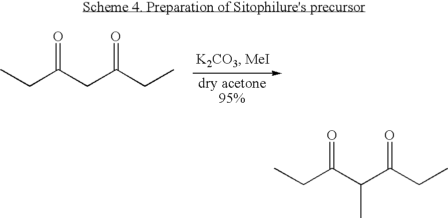

An isolated, NADPH-dependent ketoreductase was used for the synthesis of the aggregation pheromone of the pests rice weevil (Sitophilus oryzae L.) and maize weevil (Sitophilus zeamais M.). To the best of our knowledge this is the easiest and most straightforward synthesis of pheromone (+)-Sitophilure in two steps and overall yield 81%, starting from commercially available 3,5-heptanedione.

### Experimental Section

General Methods

4-Methyl-3,5-heptanedione was prepared from commercially available 3,5-heptanedione by alkylation with methyl iodide.

Racemic 5-hydroxy-4-methyl-3-heptanone was prepared from 4-methyl-3,5-heptanedione by reduction with sodium borohydride.

The progress of the enzymatic reactions and the selectivities were determined by gas chromatography (BP5890II gas chromatograph equipped with an FID detector; column: 30 m×0.25 mm×0.25 μm chiral capillary column, 20% permethylated cyclodextrin). ¹H NMR and ¹³C NMR spectra were recorded on 300, 500 MHz Bruker spectrometers in CDCl₃ solutions, using Me₄Si as an internal standard. Chemical shifts are reported in ppm downfield from Me₄Si. Yields refer to isolated and spectroscopically pure materials.

### Synthesis of 4-methyl-3,5-heptanedione

The substrate was prepared from commercially available 3,5-heptanedione according to the following procedure: Under a nitrogen atmosphere, 3,5-heptanedione (5 mmol, 640 mg, 676 μL) was dissolved in anhydrous acetone (20 mL), and pre-dried potassium carbonate (4.7 mmol, 642 mg) was added. After stirring the solution at room temperature for 5 min, methyl iodide (6.15 mmol, 873 mg, 383 μL) was added by syringe and the reaction mixture was refluxed for 20 hours. After completion of the reaction, 30 mL of diethyl ether added, the mixture was filtered, and the solvent was evaporated to dryness. Without any further purification, 4-methyl-3,5-heptanedione was subjected to enzymatic reduction. Isolated yield 95% (674 mg) in equilibrium with enolic form. ¹H NMR (CDCl₃ 300 MHz, δ ppm): 6.66 (q, J=6.9 Hz, 1H), 2.33-255 (m, 8H), 1.80 (s, 3H), 1.28 (d, J=7.2 Hz, 3H), 1.11 (t, J=7.5 Hz, 3H), 1.02 (t, J=7.2 Hz, 3H).

### Synthesis of racemic 5-hydroxy-4-methyl-3-heptanone

Under a nitrogen atmosphere, sodium borohydride (0.098 mmol, 4 mg) was added in dry ethanol (10 mL), and the mixture was cooled to 0° C. After stirring for 5 min., a solution of dry ethanol (5 mL) containing 4-methyl-3,5-heptanedione (0.3 mmol, 43 mg) was added dropwise. After stirring for 2 hours at 0° C., the reaction was quenched with saturated ammonium chloride and the ethanol was evaporated in a rotary evaporator. Then water (15 mL) was added and extracted twice with ethyl acetate (2×10 mL). The organic layer was dried over MgSO₄, and evaporated to dryness. Pure products obtained using silica gel chromatography (hexane/EtOAc, v/v, 6/1), 80% isolated yield. ¹H NMR (CDCl₃ 300 MHz, δ ppm): 3.73-3.84 (m, 1H), 3.54-3.64 (m, 1H), 2.71 (s, OH), 2.36-2.68 (m, 6H), 1.28-1.59 (m, 4H), 1.10 (d, J=7.2 Hz, 3H), 1.09 (d, J=7.2 Hz, 3H), 1.03 (t, J=7.2 Hz, 3H), 1.02 (t, J=7.2 Hz, 3H), 0.95 (t, J=7.2 Hz, 3H), 0.92 (t, J=7.2 Hz, 3H). ¹³C NMR (CDCl₃ 300 MHz, δ ppm): 216.8, 216.7, 75.0, 72.6, 50.5, 49.3, 36.0, 35.1, 27.5, 26.9, 14.2, 10.4, 9.9, 9.8, 7.6, 7.5. GC data: (column: 30 m×0.25 mm×0.25 μm chiral capillary column, 20% permethylated cyclodextrin 65° C. for 100 min, rate: 1° C./min, final temp.: 100° C.; carrier gas: N₂, press 70 kPa). t_{R}=93.3 min and 98.5 min (syn-5-hydroxy-4-methyl-3-heptanone), t_{R}=102.5 min and 115.1 min (anti-5-hydroxy-4-methyl-3-heptanone).

Enzymatic Reductions

Sixty-four different ketoreductases (KRED-101-131 and KRED-EXP-A1C; BioCatalytics, Inc., Pasadena, Calif. USA) were screened to determine the best enzymes for the selective reduction of substrate 4-methyl-3,5-heptanedione. In addition to the ketoreductases, both NADPH and glucose dehydrogenase (GTDH) are products available from BioCatalytics.

Small-Scale Enzymatic Reduction

4-methyl-3,5-heptanedione (25 mM) was mixed with NADPH (2.5 mM, 2 mg), each ketoreductase (2 mg/mL), glucose (100 mM, 18 mg), glucose dehydrogenase (GDH, 2 mg/mL) for cofactor recycling, NaCl (100 mM, 6 mg) and sodium phosphate buffer (1 mL, 200 mM, pH 6.5-6.9). The reactions were incubated at 25° C.-37° C. and reaction aliquots were taken every hour. After extraction with ethyl acetate, they were analyzed by GC chromatography.

Larger-Scale Enzymatic Reductions

### Synthesis of (4S,5S)-5-hydroxy-4-methyl-3-heptanone

A phosphate-buffered solution (20 mL, pH 6.9, 200 mM) containing 50 mM (1 mmol, 142 mg) of 4-methyl-3,5-heptanedione, NaCl (200 mM, 234 mg), glucose (120 mM, 432 mg), NADPH (0.5 mM, 0.01 mmol, 9 mg), glucose dehydrogenase (10 mg) and KRED-119 (10 mg) was stirred at 37° C. for 24 hours, until GC analysis of crude extracts showed complete reaction. Periodically the pH was readjusted to 6.9 with NaOH (2 M). The product was isolated by extracting the crude reaction mixture with EtOAc (15 mL×2). The combined organic layers were then extracted with saturated NaCl solution, dried over MgSO₄ and evaporated to dryness. Pure (4S,5S)-5-hydroxy-4-methyl-3-heptanone (125 mg) was obtained in 87% yield. ¹H NMR (CDCl₃ 300 MHz, δ ppm): 3.54-3.65 (m, 1H), 2.36-2.70 (m, 3H), 1.29-1.61 (m, 2H), 1.10 (d, J=7.2 Hz, 3H), 1.03 (t, J=7.2 Hz, 3H), 0.95 (t, J=7.2 Hz, 3H). ¹³C NMR (CDCl₃ 300 MHz, δ ppm): 216.8, 75.0, 50.5, 36.0, 27.6, 14.2, 10.0, 7.5.

Determination of the Enantiomeric Purity of (4S,5S)-5-hydroxy-4-methyl-3-heptanone: GC (column: 30 m×0.25 mm×0.25 μm chiral capillary column, 20% permethylated cyclodextrin 65° C. for 100 min, rate: 1° C./min, final temp.: 100° C.; carrier gas: N₂, press 70 kPa). t_{R}=93.3 min [<1%, (4R,5S)-5-hydroxy-4-methyl-3-heptanone], t_{R}=15.4 min [>99%, (4S,5R)-5-hydroxy-4-methyl-3-heptanone]. The enantiomeric purity was estimated to be >99% and the diastereomeric purity 99%.

### Synthesis of (4S,5R)-5-hydroxy-4-methyl-3-heptanone

A phosphate-buffered solution (16 mL, pH 6.5, 200 mM) containing 84 mM (1.35 mmol, 192 mg) of 4-methyl-3,5-heptanedione, NaCl (200 mM, 200 mg), glucose (130 mM, 375 mg), NADPH (0.69 mM, 0.011 mmol, 10 mg), glucose dehydrogenase (10 mg) and KRED-A1C (10 mg) was stirred at 25° C. for 24 hours, until GC analysis of crude extracts showed complete reaction. Periodically the pH was readjusted to 6.5 with NaOH (2 M). The product was isolated by extracting the crude reaction mixture with EtOAc (15 mL×2). The combined organic layers were then extracted with saturated NaCl solution, dried over MgSO₄ and evaporated to dryness. Pure (4S,5R)-5-hydroxy-4-methyl-3-heptanone (165 mg) was obtained in 85% yield. ¹H NMR (CDCl₃ 500 MHz, δ ppm): ¹H NMR (CDCl₃ 500 MHz, δ ppm): 3.77-3.85 (m, 1H), 2.72 (s, OH), 2.41-2.64 (m, 3H), 1.32-1.58 (m, 2H), 1.12 (d, J=7.1 Hz, 3H), 1.05 (t, J=7.3 Hz, 3H), 0.95 (t, J=7.4 Hz, 3H). ¹³C NMR (CDCl₃ 300 MHz, δ ppm): 216.7, 72.6, 49.3, 35.1, 26.9, 10.4, 9.9, 7.6.

Determination of the Enantiomeric Purity of (4S,5R)-5-hydroxy-4-methyl-3-heptanone: GC (column: 30 m×0.25 mm×0.25 μm chiral capillary column, 20% permethylated cyclodextrin 65° C. for 100 min, rate: 1° C./min, final temp.: 100° C.; carrier gas: N₂, press 70 kPa). t_{R}=100.0 min [98%, (4S,5R)-5-hydroxy-4-methyl-3-heptanone], t_{R}=105.1 min [2%, (4R,5R)-5-hydroxy-4-methyl-3-heptanone]. The enantiomeric purity was estimated to be >99% and the diastereomeric purity 96%.

Preparation of MPA-Esters

### Synthesis of (R)-MPA ester of (4S,5S)-5-hydroxy-4-methyl-3-heptanone

To a solution of (4S,5S)-5-hydroxy-4-methyl-3-heptanone (0.11 mmol, 16 mg) in dry CH₂Cl₂ were added 1.1 equiv. of DCC (0.121 mmol, 25 mg) and 1.1 equiv. of the (R)-MPA ester (0.11 mmol, 20 mg) and the reaction mixture was stirred at 0° C. for 3 hr. After completion of the reaction the produced urea was filtered, the filtrate was evaporated and then chromatographed with 5/1 Hex/EtOAc and the produced corresponding MPA-ester was isolated (27 mg). Yield 89%. ¹H NMR (CDCl₃ 500 MHz, δ ppm): 7.33-7.47 (m, 5H), 5.11 (m, 1H), 4.72 (s, 1H), 3.44 (s, 3H), 2.87 (m, 1H), 2.37-2.52 (m, 2H), 1.41-1.59 (m, 2H), 1.05 (d, J=7 Hz, 3H), 1.02 (t, J=7 Hz, 3H), 0.59 (t, J=7.5 Hz, 3H).

### Synthesis of (S)-MPA ester of (4S,5S).5-hydroxy-4-methyl-3-heptanone

To a solution of (4S,5S)-5-hydroxy-4-methyl-3-heptanone (0.056 mmol, 8 mg) in dry CH₂Cl₂ were added 1.1 equiv. of DCC (0.0616 mmol, 13 mg) and 1.1 equiv. of the (S)-MPA ester (0.0616 mmol, 10 mg) and the reaction mixture was stirred at 0° C. for 3 hr. After completion of the reaction the produced urea was filtered, the filtrate was evaporated and then chromatographed with 5/1 Hex/EtOAc and the produced corresponding MPA-ester was isolated (13 mg). Yield 87%. ¹H NMR (CDCl₃ 500 MHz, δ ppm): 7.32-7.46 (m, 5H), 5.14 (m, 1H), 4.73 (s, 1H), 3.41 (s, 3H), 2.73 (m, 1H), 2.07-2.29 (m, 2H), 1.49-1.73 (m, 2H), 0.89 (d, J=7 Hz, 3H), 0.86 (t, J=7.5 Hz, 3H), 0.83 (t, J=7.5 Hz, 3H).

### Synthesis of (R)-MPA ester of (4S,5R)-5-hydroxy-4-methyl-3-heptanone

To a solution of (4S,5R)-5-hydroxy-4-methyl-3-heptanone (0.076 mmol, 11 mg) in dry CH₂Cl₂ were added 1.1 equiv. of DCC (0.0836 mmol, 17 mg) and 1.1 equiv. of the (R)-MPA ester (0.0836 mmol, 14 mg) and the reaction mixture was stirred at 0° C. for 3 hr. After completion of the reaction the produced urea was filtered, the filtrate was evaporated and then chromatographed with 5/1 Hex/EtOAc and the produced corresponding MPA-ester was isolated (18 mg). Yield 85%. ¹H NMR (CDCl₃ 500 MHz, δ ppm): 7.33-7.48 (m, 5H), 5.12 (m, 1H), 4.76 (s, 1H), 3.44 (s, 3H), 2.65 (m, 1H), 2.17 (q, J=7 Hz, 2H), 1.52-1.62 (m, 2H), 0.88 (t, J=7.5 Hz, 3H), 0.87 (d, J=7 Hz, 3H), 0.86 (t, J=7.5 Hz, 3H).

### Synthesis of (S)-MPA ester of (4S,5R)-5-hydroxy-4-methyl-3-heptanone

To a solution of (4S,5R)-5-hydroxy-4-methyl-3-heptanone (0.125 mmol, 18 mg) in dry CH₂Cl₂ were added 1.1 equiv. of DCC (0.138 mmol, 28 mg) and 1.1 equiv. of the (S)-MPA ester (0.138 mmol, 23 mg) and the reaction mixture was stirred at 0° C. for 3 hr. After completion of the reaction the produced urea was filtered, the filtrate was evaporated and then chromatographed with 5/1 Hex/EtOAc and the produced corresponding MPA-ester was isolated (30 mg). Yield 88%. ¹H NMR (CDCl₃ 500 MHz, δ ppm): 7.31-7.48 (m, 5H), 5.15 (m, 1H), 4.76 (s, 1H), 3.44 (s, 3H), 2.78 (m, 1H), 2.36-2.57 (m, 2H), 1.45 (m, 2H), 1.05 (d, J=7 Hz, 3H), 1.03 (t, J=7.5 Hz, 3H), 0.58 (t, J=7.5 Hz, 3H).

The invention has been described with reference to various embodiments and examples, but is not limited thereto. Persons having ordinary skill in the art will appreciate that the invention can be modified in a number of ways without departing from the invention, which is limited only by the appended claims and equivalents thereof.

What is claimed:

1. A method for producing (+)-Sitophilure ((4R,5S)-5-hydroxy-4-methyl-3-heptanone), comprising:
contacting 4-methyl-3,5-heptadione with a reduced nicotinamide cofactor and a ketoreductase enzyme capable of catalyzing the reduction of 4-methyl-3,5-heptadione to produce (4R,5S)-5-hydroxy-4-methyl-3-heptanone to the substantial exclusion of other diastereomers.

2. The method of claim 1, wherein the reduced nicotinamide cofactor is NADPH.

3. The method of claim 1, wherein the nicotinamide cofactor is recycled.

4. The method of claim 1, wherein the (4R,5S)-5-hydroxy-4-methyl-3-heptanone is produced in greater than 90% diasteromeric excess.

5. The method of claim 1, wherein the (4R,5S)-5-hydroxy-4-methyl-3-heptanone is produced in greater than 98% diasteromeric excess.

6. A method for producing (+)-Sitophilure ((4R,5S)-5-hydroxy-4-methyl-3-heptanone), comprising:
methylating 3,5-heptanedione to produce 4-methyl-3,5-heptadione; and
contacting said 4-methyl-3,5-heptadione with a reduced nicotinamide cofactor and a ketoreductase enzyme capable of catalyzing the diastereoselective reduction of 4-methyl-3,5-heptadione to produce (4R,5S)-5-hydroxy-4-methyl-3-heptanone.

7. The method of claim 6, wherein the nicotinamide cofactor is recycled.

8. The method of claim 6, wherein the reduced nicotinamide cofactor is NADPH.

9. A method for producing (4S,5S)-5-hydroxy-4-methyl-3-heptanone, comprising:
contacting 4-methyl-3,5-heptadione with a reduced nicotinamide cofactor and a ketoreductase enzyme capable of catalyzing the reduction of 4-methyl-3,5-heptadione to produce (4S,5S)-5-hydroxy-4-methyl-3-heptanone to the substantial exclusion of other diastereomers.

10. The method of claim 9, wherein the (4S,5S)-5-hydroxy-4-methyl-3-heptanone is produced in at least 99% diasteromeric excess.

11. A method for producing (4S,5R)-5-hydroxy-4-methyl-3-heptanone, comprising:
contacting 4-methyl-3,5-heptadione with a reduced nicotinamide cofactor and a ketoreductase enzyme capable of catalyzing the reduction of 4-methyl-3,5-heptadione to produce (4S,5R)-5-hydroxy-4-methyl-3-heptanone to the substantial exclusion of other diastereomers.

12. The method of claim 11, wherein the (4S,5R)-5-hydroxy-4-methyl-3-heptanone is produced in at least 96% diasteromeric excess.
